(19) United States
(12) Patent Application Publication
(10) Pub. No.: US 2010/0245765 A1
(43) Pub. Date: Sep. 30, 2010

(54) VIDEO INFRARED OPHTHALMOSCOPE

(75) Inventors: David S. Dyer, Olathe, KS (US); James Higgins, Overland Park, KS (US)

Correspondence Address: STAAS & HALSEY LLP, SUITE 700, 1201 NEW YORK AVENUE, N.W., WASHINGTON, DC, 20005, US

(73) Assignee: Dyer Holdings, LLC, Olathe, KS (US)

(21) Appl. No.: 12/606,868

(22) Filed: Oct. 27, 2009

Related U.S. Application Data
(60) Provisional application No. 61/109,034, filed on Oct. 28, 2008.

(51) Int. Cl. A61B 3/12

(52) U.S. Cl. 351/206; 351/246

(57) ABSTRACT

An opthalmoscope includes a wearable headset. The wearable headset has a light source, a beam splitter reflecting infrared radiation from the light source to an eye, a camera collecting radiation reflected by the eye through the beam splitter, an analog to digital convertor receiving a raw signal from the camera based on the collected radiation, the analog to digital convertor converting the raw signal to a digital signal; a black and white to color converter converting the digital signal into a color signal, a streaming video converter processing the color signal into a video signal, and a pair of video monitors displaying an image of the eye based on the video signal. The wearable headset also has a video transmitter, the video transmitter transmitting the video signal to a computer over a network, the computer extracting a plurality of images from the video signal.

### CROSS-REFERENCE TO RELATED APPLICATIONS

This application is based upon and claims the benefit of priority to U.S. Application No. 61/109,034; filed on Oct. 28, 2008, the entire contents of which are incorporated by reference herein.

### BACKGROUND

1. Field

A video infrared opthalmoscope uses an infrared light to illuminate an ocular system and a camera to capture and display an image. The image may be analyzed and processed, and rendered in 3-D.

2. Description of the Related Art

An opthalmoscope is used to look into a patient's eye and view the intra-ocular contents. Specifically, an opthalmoscope is used to view a retina, cornea, iris, lens, and vitreous within the eye.

### SUMMARY

In one aspect, an indirect opthalmoscope includes a wearable headset, and the wearable headset includes a light source, a beam splitter reflecting infrared radiation from the light source to an eye, a camera collecting radiation reflected by the eye through the beam splitter, an analog to digital convertor receiving a raw signal from the camera based on the collected radiation, the analog to digital convertor converting the raw signal to a digital signal; a black and white to color converter converting the digital signal into a color signal, a streaming video converter processing the color signal into a video signal, and a pair of video monitors displaying an image of the eye based on the video signal, the wearable headset also including a video transmitter, the video transmitter transmitting the video signal to a computer over a network, the computer extracting a plurality of images from the video signal.

In another aspect, a direct opthalmoscope includes a light source, a beam splitter reflecting infrared radiation from the light source through one of a plurality of focusing lenses to an eye, a camera collecting radiation reflected by the eye through the beam splitter, an analog to digital convertor receiving a raw signal from the camera based on the collected radiation, the analog to digital convertor converting the raw signal to a digital signal; a black and white to color converter converting the digital signal into a color signal, a streaming video converter processing the color signal into a video signal, and a video monitor displaying an image of the eye based on the video signal, the direct opthalmoscope also including a video transmitter, the video transmitter transmitting the video signal to a computer over a network, the computer extracting a plurality of images from the video signal.

In another aspect, a method of scanning an eye includes providing a light source, emitting infrared radiation from the light source toward a beam splitter, reflecting the infrared radiation with the beam splitter through a focusing lens, focusing the infrared radiation with the focusing lens on the eye, collecting radiation reflected by the eye through the beam splitter at a camera, producing an image signal representative of an image of the eye with the camera based on the collected radiation, and displaying the image of the eye produced by the image signal on a display.

The above-described embodiments of the present invention are intended as examples, and all embodiments of the present invention are not limited to including the features described above.

### BRIEF DESCRIPTION OF THE DRAWINGS

FIG. 1 shows a wireless direct infrared opthalmoscope according to an embodiment of the invention;

FIG. 2 shows a wired direct infrared opthalmoscope according to an embodiment of the invention;

FIG. 3 shows a wired indirect infrared opthalmoscope according to an embodiment of the invention;

FIG. 4 shows a wireless indirect infrared opthalmoscope according to an embodiment of the invention;

FIG. 5 shows a ray diagram for use with an infrared opthalmoscope;

FIG. 6 shows a ray diagram for use with an infrared opthalmoscope;

FIGS. 7A and 7B show a three-quarter view, partially cut away, of a wireless indirect infrared opthalmoscope according to an embodiment of the invention; and

FIG. 8 shows a pulse width modulated power supply for use with an embodiment of the invention.

### DETAILED DESCRIPTION OF THE PREFERRED EMBODIMENTS

Reference may now be made in detail to embodiments of the present invention, examples of which are illustrated in the accompanying drawings, wherein like reference numerals refer to like elements throughout.

An opthalmoscope is used by an ophthalmologist to examine a patient's eyes. The light from the opthalmoscope must pass through the pupil of the eye to reach the retina. Since a pupil is sensitive to light, and constricts in the presence of light, shining visible light into the eye makes it more difficult to perform the examination, since the pupil constricts, and restricts a view of the retina. Ophthalmologists can take steps to dilate the pupil, such as by using eye drops, and performing the examination in a dark room. Some patients may be allergic to eye drops, however. Eye drops also require a recovery time, and a certain amount of time to begin to work.

In situations such as emergencies, when time is precious and every second counts, there may not be time to call in a specialist. It would be advantageous if an image of an eye could be captured and sent to the specialist remotely, during an examination of the eye. It would be further advantageous if an examination of the eye could be conducted by on the spot personnel under the guidance of a specialist who is not at the scene, but who was viewing images of the eye remotely.

Since an ophthalmologist generally holds a small focusing lens in front of the eye, the image of the eye appears to him to be upside down and backwards. It takes significant training and adjustment to get used to looking at an image upside down and backwards. For example, if the ophthalmologist wants to view the right side of the eye, the lens is moved to the left. It would be advantageous if an image that was right side up and frontwards were presented to the ophthalmologist.

Opthalmoscopes generally include a lamp with a power supply to provide illumination. A lamp such as a halogen bulb lamp that produces visible light has been used. Opthalmoscopes also include reflectors to redirect the light from the lamp to the retina, while allowing returning reflected light to pass and create the image for the ophthalmologist to view. It would be advantageous if the ophthalmologist could capture the image for later viewing. It would be advantageous if a video of the examination of the retina could be created, and analyzed at a later time, or stored for reference. Such a reference video could be used to form a baseline to evaluate whether the condition of the retina is changing and, if so, how fast. For example, if a video of the examination were captured, a video taken at a later date could be compared to it, or even overlaid, and scaled to match. Then areas of the two videos that deviated from one another could be evaluated easily.

It would also be advantageous if multiple video images could be combined to produce a montage of the entire retina and two dimensional or three-dimensional spaces. The retina could be viewed from different angles, for example, and the different angles of view could be combined to create a 3-D image. It would also be advantageous if the image of the retina could be rotated, and cross-sectional slices could be extracted from the images. It would also be advantageous if lesions and other artifacts could be measured for comparison with videos taken during earlier or later examinations. It would also be advantageous if specific tissues could be colorized for easier identification.

It would also be advantageous if the video images of the retinal examination could be collected in real time, and transmitted over a network to other locations. This way, an examination performed at a remote location, for example a location without specialized medical personnel, could be reviewed by an ophthalmologist. A retinal examination could be performed in the field by general practitioners, under the direction of an ophthalmologist who was reviewing video of the examination remotely. A retinal examination could be reviewed by other personnel in a hospital setting, such as in the same medical facility or hospital at which the will examination is being performed. This would allow medical schools for example, to allow medical students to see an examination being conducted for learning purposes. It would also allow real-time video of the examination to be collected for insurance or litigation purposes, such as evidence that a certain standard of care was maintained.

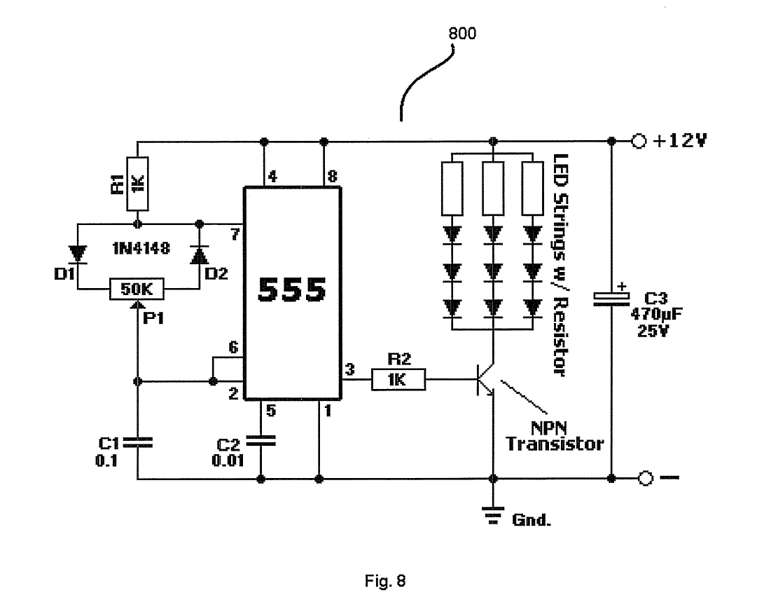

Since many light emitting diodes (LED) are current devices, the degree to which they illuminate is proportional to the amount of electric current flowing through the LED, rather than on the voltage drop across it. Standard voltage varying supplies may not be efficient or linear when trying to control light intensities emitted by the light emitting diode. It would be advantageous if a power supply took advantage of the very fast turn on times of LEDs (typically measured in nano-seconds) by turning the LED on and off very rapidly with a pulsed width square wave, allowing for control of light intensity. In FIG. 8 is shown a pulse width modulated power supply 800 for use with an embodiment of the invention.

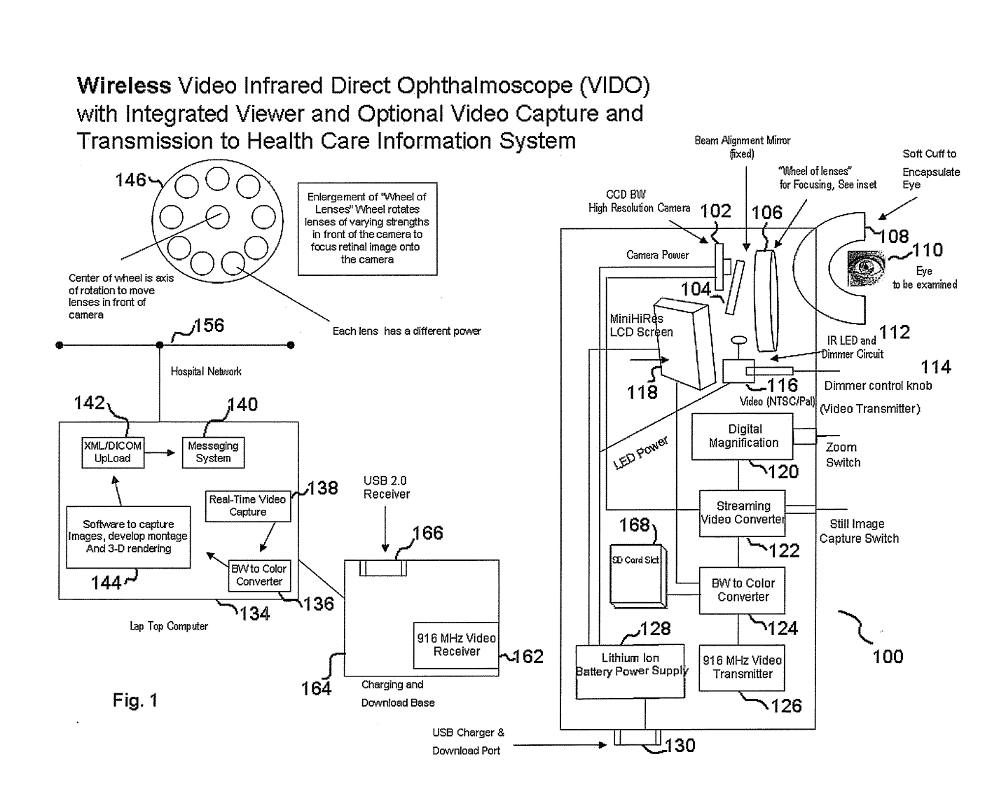

In FIG. 1 is shown a wireless direct infrared opthalmoscope 100 according to an embodiment of the invention. The direct infrared opthalmoscope 100 may be used by an internist, a family practitioner, or an emergency physician to visualize the intraocular contents of the eye during a physical examination. The direct infrared opthalmoscope 100 is held in front of the physician's eye by a handle, and then moved forward toward the patient's eye until the visible light is adjusted to visualize inside the patient's eye.

The direct infrared opthalmoscope 100 has a light source 116, which may be an infrared light source, such as a light emitting diode (LED). In other embodiments, the light source 116 may be an electric lamp, a mercury vapor lamp, a halogen lamp, or a tungsten filament lamp. The light source 116 may be equipped with a filter to filter out visible wavelengths and pass infrared wavelengths of radiation. The light source 116 has a dimmer switch 112. The light source 116 may be powered by a power supply 128. The power supply 128 may be a battery, such as a rechargeable lithium ion battery, a nickel cadmium battery, or an alkaline battery.

The light source 116 emits radiation in the range of 800-950 nm, and particularly, at about 945 nm. The dimmer switch 112 controls the intensity of the infrared radiation emitted by the light source 116, such as by a rheostat, or an amplifier. The dimmer circuit may be controlled by a dimmer control knob 114. The ophthalmologist may manipulate the dimmer control knob 114 during the examination to increase or reduce the amount of infrared radiation shed on the patient's eye.

In one embodiment, the light source 116 is a light emitting diode. Since light emitting diodes are current devices, the degree to which they illuminate is proportional to the amount of electric current flowing through the light emitting diode, rather than to the voltage drop across the light emitting diode. Consequently, a power supply that varies voltage across the light source 116 may not be efficient or linear when trying to control the intensity of radiation emitted from light source 116. In one embodiment, the intensity of radiation produced by the light source 116 is controlled by supplying a pulsed width square wave to turn the light source 116 on and off very rapidly. Since a light emitting diode has a very fast turn on time, typically measured in nano-seconds, the intensity of radiation emitted from the light source 116 can be varied by varying the width of the pulses supplied to the light source 116.

Radiation from the light source 116 may be focused through a lens 106 toward the eye to be examined 110. In one embodiment, the lens 106 may be an adjustable positive or negative diopter focusing lens. The lens 106 may be one of a plurality of lens in a wheel of focusing lenses 146 of varying powers. The wheel of focusing lenses 146 may be rotated to select the proper lens for examination.

The direct infrared opthalmoscope 100 may be equipped with a soft cuff 108 to encapsulate the eye to be examined 110. The soft cuff 108 may be disposable to prevent contamination between patients. The soft cuff 108 rests on the forehead and cheek to completely cover the orbit surrounding the eye, and keep ambient, or background light from interfering with the examination. The radiation from the light source 116 through the lens 106 also passes through the soft cuff 108 to reach the eye to be examined 110. In one embodiment, the soft cuff 108 may be inflatable. The soft cuff 108 keeps the direct infrared opthalmoscope 100 in a stable position close to the eye to be examined 110, and limits movement between the direct infrared opthalmoscope 100 and the eye to be examined 110, without the need for the observer to be close to the patient. The soft cuff 108 also allows the pupil to dilate naturally, to afford a better view inside the eye, by blocking out substantially all of the surrounding light. Consequently, in one embodiment, an examination of the eye using the direct infrared opthalmoscope 100 can be performed in a room with normal lighting.

Radiation reflected by the eye to be examined 110 returns through an aperture in the soft cuff 108 and the lens 106 and through the beam splitter 104. In one embodiment, the beam splitter 104 may be fixed in place. The radiation passes through the beam splitter 104 and is collected by a camera 102, such as a high-resolution camera 102. The camera 102 is powered by the power supply 128 as well.

In one embodiment, the camera 102 is a charge coupled device. In another embodiment, the camera 102 is a complementary metal-oxide-semiconductor (CMOS) based device, or an array of light emitting diodes running in reverse, i.e., collecting light and converting it into an electrical signal. The camera 102 may be a black-and-white camera. An autofocus lens may be mounted in front of the camera 102 to focus the light returning from the eye to be examined 110. In another embodiment, visible light is used to examine the eye, and in that case, the image may be captured by a color camera.

The camera 102 captures an image of the eye to be examined 110 formed by the infrared radiation. A video signal formed by the camera 102 of the image of the eye to be examined 110 is converted from an analog signal to a digital signal by a streaming video converter 122. In the event that the camera 102 is a digital camera, such as a digital charge coupled device, then no converter is needed. The image of the eye may also be magnified in a magnifier 120, such as a digital magnifier 120 after the signal is converted to a digital signal.

Next, the signal may be converted from black-and-white, or grayscale, to color in a black-and-white to color converter 124. In one embodiment, the black-and-white to color converter 124 maps intensities of pixels of a charge coupled device to separate colors. Mapping the intensities of the pixels to colors may include interpolating pixel intensities between two (or more) pixels, or extrapolating pixel intensities around edges.

In one embodiment, the black-and-white to color converter 124 creates a map of grey scale to color that is appropriate for the various eye structures, like blood vessels, the optic nerve, and fovea. In this embodiment, the black-and-white to color converter 124 may normalize the black-and-white image of the eye. The image of the eye may be normalized with a histogram normalizer. Normalizing the image of the eye produces a uniform intensity profile of the image. The black-and-white to color converter 124 may also use edge detection image processing to identify the blood vessels and other structures of the eye. Finally, after the image of the eye has been mapped, direct spatial domain intensity transformations are applied to each structure of the eye, resulting in a colorized image of the eye.

The black-and-white to color converter 124 may also work by scaling wavelength components in the infrared range by a predetermined amount so that the wavelengths are mapped to the visible range instead.

The image signal is then sent to a screen 118 to display the image for the observer, so that the observer may view the images from inside the patient's eye. In one embodiment, the image is manipulated so that it is right side up and frontwards when it is presented to the ophthalmologist.

In one embodiment, the screen 118 may be a high-resolution liquid crystal display screen. In another embodiment, the screen 118 may be an array of light emitting diodes or a plasma display screen. A lens, such as a high focus or positive diopter lens may be mounted over the screen 118. Such a lens mounted over the screen 118 may magnify the image and limit the accommodation necessary to focus on the screen 118. The screen 118 may receive power from the power supply 128.

The signal from the streaming video converter 122 may also be sent to a video transmitter 126, which transmits the image signal over a wireless connection to a laptop computer 134 for documentation and storage. In one embodiment, the video transmitter 126 transmits the image signal to a video receiver 162 coupled to the laptop computer 134. In one embodiment, the video transmitter 126 transmits in the range of 800-1000 MHz, such as at 916 MHz.

In one embodiment, the video transmitter 126 sends a raw digital video signal to a laptop computer 134. In this embodiment, the laptop computer 134 has a separate black-and-white to color converter 136, as well as a real-time video capture 138. The real-time video capture 138 captures the video signal in real time, and sends it to the black-and-white to color converter 136. Software 144 on the laptop may be used to manipulate the signal by capturing the raw image, adjusting the contrast, white balance, black balance, color saturation, or brightness. Separate images of the eye can be “stitched” together to form a montage. A three-dimensional image can be developed from the images as well. A three-dimensional image can be rotated or manipulated, such as translated in the X, Y, or Z axes. Cross-sectional images can be produced from the separate images as well.

Images of lesions or tumors found during an examination can be measured and compared using the images taken during sequential examination visits. In one embodiment, the images conform to the XML, JPEG or DICOM standards. In another embodiment; the images conform to an MPEG-4 standard. Images and files produced by the direct infrared opthalmoscope 100 may be able to interface with any hospital or medical record software system. Downloads of the images and associated data over a network to the medical record system may be allowed. Connections to the network can be wireless or wired. Remote access to the images on the laptop or on the medical record software may be allowed by software.

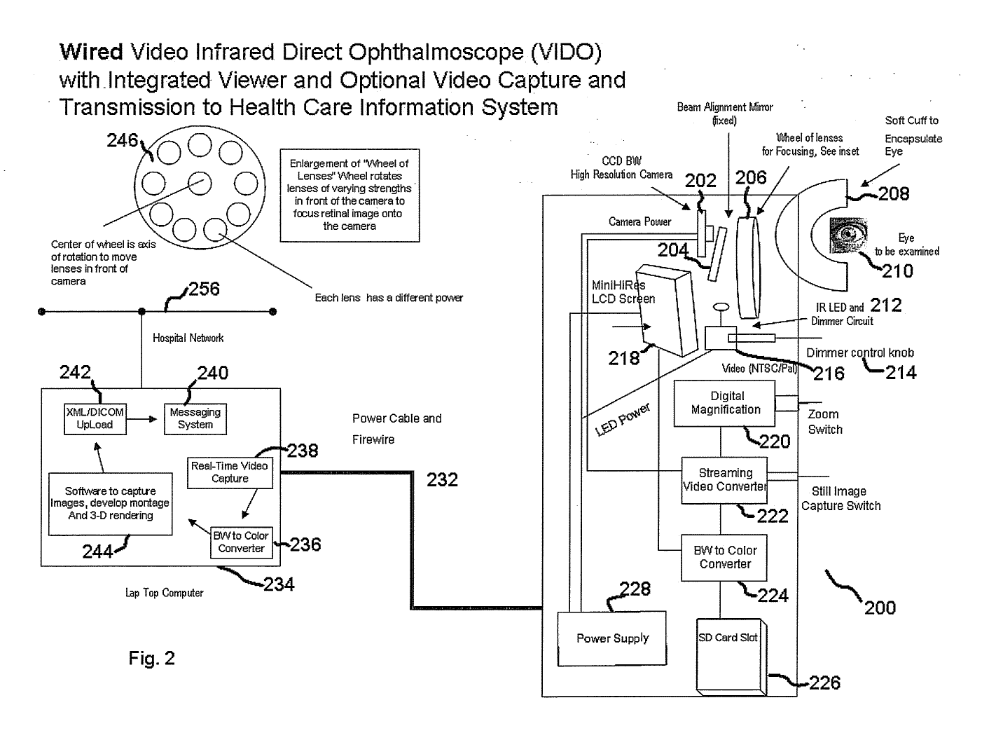

In FIG. 2 is shown a wired direct infrared opthalmoscope 200 according to an embodiment of the invention. The direct infrared opthalmoscope 200 may be used by an internist, a family practitioner, or an emergency physician to visualize the intraocular contents of the eye during a physical examination. The direct infrared opthalmoscope 200 is placed in front of the physician's eye, and then moved forward toward the patient's eye until the visible light is adjusted to visualize inside the patient's eye.

The direct infrared opthalmoscope 200 has a light source 216, which may be an infrared light source, such as a light emitting diode (LED). In other embodiments, the light source 216 may be an electric lamp, a mercury vapor lamp, a halogen lamp, or a tungsten filament lamp. The light source 216 may be equipped with a filter to filter out visible wavelengths and pass infrared wavelengths of radiation. The light source 216 has a dimmer switch 212. The light source 216 may be powered by a power supply 228. The power supply 228 may be a battery, such as a rechargeable lithium ion battery, a nickel cadmium battery, or an alkaline battery.

The light source 216 emits radiation in the range of 800-950 nm, and particularly, at about 945 nm. The dimmer switch 212 controls the intensity of the infrared radiation emitted by the light source 216, such as by a rheostat, or an amplifier. The dimmer circuit may be controlled by a dimmer control knob 214. The ophthalmologist may manipulate the dimmer control knob 214 during the examination to increase or reduce the amount of infrared radiation shed on the patient's eye.

In one embodiment, the light source 216 is a light emitting diode. Since light emitting diodes are current devices, the degree to which they illuminate is proportional to the amount of electric current flowing through the light emitting diode, rather than to the voltage drop across the light emitting diode. Consequently, a power supply that varies voltage across the light source 216 may not be efficient or linear when trying to control the intensity of radiation emitted from light source 216. In one embodiment, the intensity of radiation produced by the light source 216 is controlled by supplying a pulsed width square wave to turn the light source 216 on and off very rapidly. Since a light emitting diode has a very fast turn on time, typically measured in nano-seconds, the intensity of radiation emitted from the light source 216 can be varied by varying the width of the pulses supplied to the light source 216.

Radiation from the infrared light emitting diode 212 may be focused through a lens 206 toward the eye to be examined 210. In one embodiment, the lens 206 may be an adjustable positive or negative diopter focusing lens. The lens 206 may be one of a plurality of lens in a wheel of focusing lenses 246 of varying powers. The wheel of focusing lenses 246 may be rotated to select the proper lens for examination.

The direct infrared opthalmoscope 200 may be equipped with a soft cuff 208 to encapsulate the eye to be examined 210. The soft cuff 208 may be disposable to prevent contamination between patients. The soft cuff 208 rests on the forehead and cheek to completely cover the orbit surrounding the eye, and keep ambient, or background light from interfering with the examination. The radiation from the infrared light emitting diode 212 through the lens 206 also passes through the soft cuff 208 to reach the eye to be examined 210. In one embodiment, the soft cuff 208 may be inflatable. The soft cuff 208 keeps the direct infrared opthalmoscope 200 in a stable position close to the eye to be examined 210, and limits movement between the direct infrared opthalmoscope 200 and the eye to be examined 210, without the need for the observer to be close to the patient. The soft cuff 208 also allows the pupil to dilate naturally, to afford a better view inside the eye, by blocking out substantially all of the surrounding light. Consequently, in one embodiment, an examination of the eye using the direct infrared opthalmoscope 200 can be performed in a room with normal lighting.

Radiation reflected by the eye to be examined 210 returns through the soft cuff 208 and the lens 206 through the beam splitter 204. In one embodiment, the beam splitter 204 may be fixed in place. The radiation passes through the beam splitter 204 and is collected by a camera 202, such as a high-resolution camera 202. The camera 202 is powered by the power supply 228 as well.

In one embodiment, the camera 202 is a complementary metal-oxide-semiconductor (CMOS) based device, or a charge coupled device. In another embodiment, the camera 202 is an array of light emitting diodes running in reverse, i.e., collecting light and converting it into an electrical signal. The camera 202 may be a black-and-white camera. In another embodiment, visible light is used to examine the eye, and in that case, the image may be captured by a color camera. An autofocus lens may be mounted in front of the camera 202 to focus the light returning from the eye to be examined 210.

The camera 202 captures an image of the eye to be examined 210 formed by the infrared radiation. A video signal formed by the camera 202 of the image of the eye to be examined 210 is converted from an analog signal to a digital signal by a streaming video converter 222. In the event that the camera 202 is a digital camera, such as a digital charge coupled device, then no converter is needed. The image of the eye may also be magnified in a magnifier 220, such as a digital magnifier 220 after the signal is converted to a digital signal. Next, the signal may be converted from black-and-white to color in a black-and-white to color converter 224. The black-and-white to color 224 may work in a manner similar to that of the black-and-white to color converter 104 shown in FIG. 1.

The image signal is then sent to a screen 218 to display the image for the observer, so that the observer may view the images from inside the patient's eye. In one embodiment, the screen 218 may be a high-resolution liquid crystal display screen. In another embodiment, the screen 218 may be an array of light emitting diodes or a plasma display screen. In one embodiment, the image is manipulated so that it is right side up and frontwards when it is presented to the ophthalmologist. A lens, such as a high focus or positive diopter lens may be mounted over the screen 218. Such a lens mounted over the screen 218 may magnify the image and limit the accommodation necessary to focus on the screen 218. The screen 218 may receive power from the power supply 228.

The signal from the streaming video converter 222 may also be sent over a wired connection 232 to a lap top computer 234 for documentation and storage. In one embodiment, the connection 232 is a Universal Serial Bus. In one embodiment, a raw digital video signal is sent to the laptop computer 234. In this embodiment, the laptop computer 234 has a separate black-and-white to color converter 236, as well as a real-time video capture 238. The real-time video capture 238 captures the video signal in real time, and sends it to the black-and-white to color converter 236. Software 244 on the laptop may be used to manipulate the signal by capturing the raw image, adjusting the contrast, white balance, black balance, color saturation, or brightness. Separate images of the eye can be “stitched” together to form a montage. A three-dimensional image can be developed from the images as well. A three-dimensional image can be rotated or manipulated, such as translated in the X, Y, or Z axes.

Images of lesions or tumors found during an examination can be measured and compared using the images taken during sequential examination visits. In one embodiment, the images conform to the JPEG or DICOM standards. In another embodiment, the images conform to an MPEG-4 standard. Images and files produced by the direct infrared opthalmoscope 200 may be able to interface with any hospital or medical record software system. Downloads of the images and associated data over a network to the medical record system may be allowed. Connections to the network can be wireless or wired. Remote access to the images on the laptop or on the medical record software may be allowed by software.

In one embodiment, images taken by the infrared direct opthalmoscope 200 may be stored on an internal memory chip, such as a SD card 226.

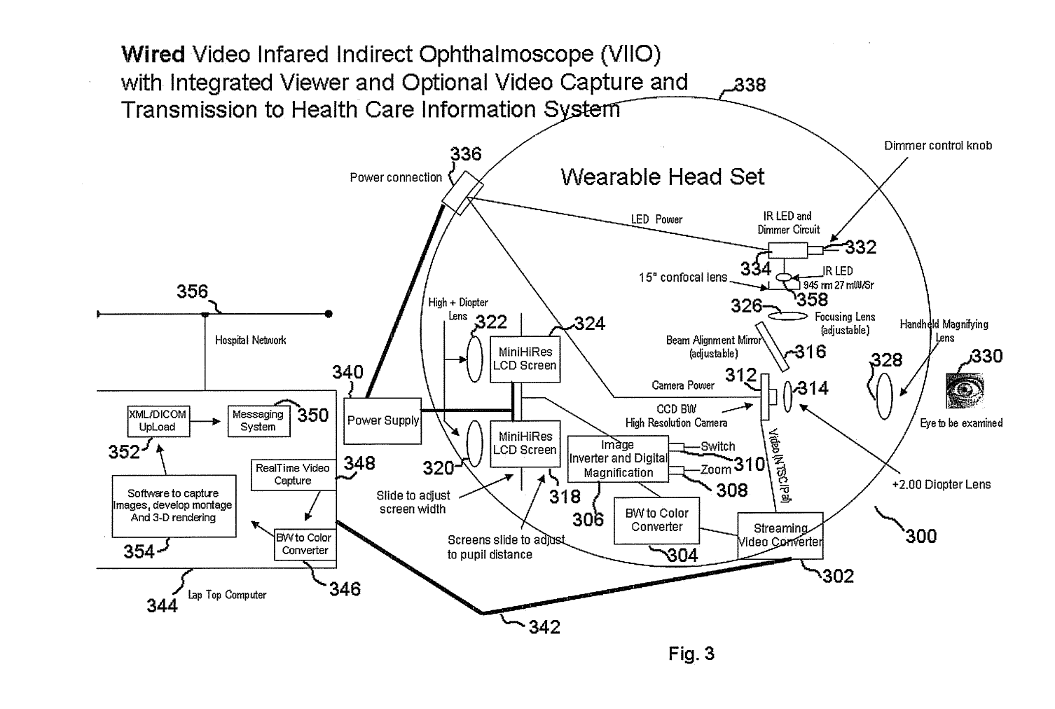

In FIG. 3 is shown a wired indirect infrared opthalmoscope 300 according to an embodiment of the invention. In one embodiment, the indirect opthalmoscope 300 may be a binocular opthalmoscope. In this case, two viewing screens 318 and 324 could be used, one for each eye, as in a pair of binoculars. In the indirect opthalmoscope 300, light is not shined directly on the ocular system. Rather, the light is diverted by at least one reflector or beam splitter. The indirect opthalmoscope 300 may include a wearable headset 338. In one embodiment, an ophthalmologist places the wearable headset 338 on his head during the examination. Wearing the wearable headset 338 including the indirect opthalmoscope 300 on the head allows light to be directed on the point to be viewed, while leaving the hands free for other functions. A wearable headset 338, in particular, allows an ophthalmologist to direct the light by turning the head in the direction in which the ophthalmologist wants to look.

In another embodiment, the wearable headset 338 is a pair of glasses or goggles.

The ophthalmologist also holds a lens 328 to direct the light from the opthalmoscope onto a patient's eye to obtain a virtual image of the retina. The lens 328 focuses the light from the opthalmoscope. The light is directed into the patient's eye through the pupil, illuminating the retina inside the eye. The light reflected by the retina is reflected out of the patient's eye, and an indirect image is formed between the lens 328, which is held in front of the patient's eye, and the ophthalmologist's eye.

The lens 328 reverses the image of the retina. The image of the retina, for example, is upside down and backwards as viewed by the ophthalmologist. This may pose a problem for an inexperienced practitioner, or one who is not used to viewing an upside down image. For example, if one wishes to move the virtual image to the right, the opthalmoscope is moved to the left. Thus, in the case of the wearable headset 338, if the ophthalmologist wanted to see the right side of the retina, the ophthalmologist would move his head to the left. This is counterintuitive, and may pose a problem to someone who is new, or not used to using an opthalmoscope. It would be advantageous if an ophthalmologist could move in the same direction that the virtual image is intended to move. It would also be advantageous if the opthalmoscope could convert the upside down and backward image to an image that simulates a real image.

As shown in FIG. 3, an embodiment of a wearable headset 338 employs a light source 334 to illuminate the retina. The light source 334 may be an infrared light source, such as a light emitting diode (LED). In other embodiments, the light source 334 may be an electric lamp, a mercury vapor lamp, a halogen lamp, or a tungsten filament lamp. The light source 334 may be equipped with a filter to filter out visible wavelengths and pass infrared wavelengths of radiation.

The light source 334 may be powered by a power supply 340, either directly or through a power connection 336. The power supply 340 may be a battery, such as a rechargeable lithium ion battery, a nickel cadmium battery, or an alkaline battery. The light source 334 emits light of an invisible nature, i.e. light that is out of the visible range, and to which the eye is not generally sensitive. The infrared light may be of a wavelength in the range of 945 nm, for example. Since the light is invisible, the pupil does not constrict when it receives the infrared light, and the examination can be performed without artificial dilation. Thus, a patient who is allergic to eye drops does not need to have them put in their eyes. Also, since artificial dilation is not necessary to perform the examination with invisible light, an examination can be performed more quickly in, for example, an emergency situation.

The output of the light source 334 may be controlled by a dimmer circuit 332, such as a rheostat, or a solid-state device like a transistor. The ophthalmologist, for example, may be able to raise or lower the amount of infrared radiation emitted by the light source 334 during examination, as the need for infrared light changes.

In one embodiment, the light source 334 is a light emitting diode. Since light emitting diodes are current devices, the degree to which they illuminate is proportional to the amount of electric current flowing through the light emitting diode, rather than to the voltage drop across the light emitting diode. Consequently, a power supply that varies voltage across the light source 334 may not be efficient or linear when trying to control the intensity of radiation emitted from light source 334. In one embodiment, the intensity of radiation produced by the light source 334 is controlled by supplying a pulsed width square wave to turn the light source 334 on and off very rapidly. Since a light emitting diode has a very fast turn on time, typically measured in nano-seconds, the intensity of radiation emitted from the light source 334 can be varied by varying the width of the pulses supplied to the light source 334.

The light source 334 might also comprise a lamp, such as a halogen lamp that emits light in other wavelengths besides infrared. In this case, the light source 334 might include a filter that filters the wavelengths outside of the infrared band so that they do not reach the pupil and cause it to constrict. In one embodiment, the filter might take the form of a beam splitter that reflects visible radiation while allowing infrared radiation to pass.

The infrared radiation from the light source 334 may pass through a lens 358, which may be a confocal lens, and an adjustable diopter focusing lens 326 to obtain a uniform beam of light. In one embodiment, the lens 358 is a 10°-35° confocal lens. In one embodiment, the focusing lens 326 may be adjustable, for varying a focal length and focusing the infrared radiation on a beam splitter 316. The beam splitter 316 may be adjustable as well, reflecting the infrared radiation through the handheld magnifying lens 328 toward the eye to be examined 330. In one embodiment, the beam splitter 360 can be adjusted to move the light beam up and down, to focus the light on the eye to be examined 330.

In one embodiment, the beam splitter 316 may be a filter, passing visible radiation and reflecting infrared radiation through the handheld magnifying lens 328 and toward the eye to be examined 330. In another embodiment, the beam splitter 316 may be a 50/50 filter, passing half of the infrared radiation while reflecting the other half of the infrared radiation through the handheld magnifying lens 328 and toward the eye to be examined 330. This may be useful if, for example, the light source 334 emits more infrared radiation that is necessary for the examination. Other proportions than 50/50 may be used, the beam splitter could be a 60/40 filter, or a 70/30 filter, as well, and in the reverse proportions too.

A virtual image is produced by the handheld lens 328 between the handheld lens 328 and the wearable headset 338. The image is captured by the camera 312, after being focused on the camera 312 by a positive diopter lens 314, such as a positive 20 diopter lens.

The camera 312 may be powered by the power supply 340, either directly or through the power connection 336. The camera 312 may collect the infrared radiation reflected by the eye to be examined, and form an image, such as a black-and-white image. In one embodiment, the camera 312 could be a charge coupled device (CCD). In another embodiment the camera 312 is a complementary metal-oxide-semiconductor (CMOS) based device, or an array of LEDs working in reverse, i.e., collecting light and converting it into an electrical signal. In one embodiment, the camera 312 could be a high-resolution camera.

The images formed by the camera 312 may be sent to a streaming video converter 302. The streaming video converter 302 may receive power from the power supply 340, either directly or through the power connection 336. The streaming video converter may process the images from the camera 312, creating a streaming video from individual still images. In one embodiment, the images from the camera 312 are converted from an analog signal to a digital signal in the streaming video converter 302. In another embodiment, camera 312 is a digital CCD camera, and so no digital conversion is necessary. In one embodiment, the streaming video converter 302 may include a computer processor to perform the analog to digital conversion, and other processing as necessary. The analog to digital conversion may be performed in software.

From the streaming video converter 302 the images may be sent to a black-and-white to color converter 304. In an alternative embodiment, the images from the camera 312 may be sent to the black-and-white to color converter 304 directly. The black-and-white to color converter 304 may receive power from the power supply 340, either directly or through the power connection 336.

In one embodiment, the black-and-white to color converter 304 maps intensities of pixels of a charge coupled device to separate colors. Mapping the intensities of the pixels to colors may include interpolating pixel intensities between two (or more) pixels, or extrapolating pixel intensities around edges.

In one embodiment, the black-and-white to color converter 304 creates a map of grey scale to color that is appropriate for the various eye structures, like blood vessels, the optic nerve, and fovea. In this embodiment, the black-and-white to color converter 304 may normalize the black-and-white image of the eye. The image of the eye may be normalized with a histogram normalizer. Normalizing the image of the eye produces a uniform intensity profile of the image. The black-and-white to color converter 304 may also use edge detection image processing to identify the blood vessels and other structures of the eye. Finally, after the image of the eye has been mapped, direct spatial domain intensity transformations are applied to each structure of the eye, resulting in a colorized image of the eye.

The black-and-white to color converter 304 may, in another embodiment, scale the various wavelengths of infrared light represented by the image from the streaming video converter 302, so that wavelengths within the visible range are formed. Wavelengths in the range of about 380 to 750 nm, for example, may be visible to the human eye. From these scaled wavelengths a visible image could be formed, in which colors were assigned to various features of the eye under examination. In one embodiment, shades of gray are assigned to the scaled wavelengths, creating a grayscale image. In another embodiment, colors of the visible spectrum are assigned to the scaled wavelengths.

If the light source 334 produces light in the near infrared range of slightly longer wavelengths than 750 nm, for example, the various wavelengths of light composing the infrared image could be scaled by a suitable scalar so they all entered the visible range in proportionally the same amount. Strictly by way of a non-limiting example, a wavelength of 945 nm, for example, in the infrared range could be scaled by 300 nm to 645 nm in the visible range. Consequently, a feature of the eye that reflects light at the wavelength of 945 nm would show up in the visible range as a particular “color.” Other wavelengths in the near infrared could be scaled in approximately the same amount. Light reflected by the feature in the range of about 950 nm could be scaled in the same proportion as the 945 nm light, so that it assumes a wavelength of 650 nm in the visible range.

Images produced by the black-and-white to color converter 304 may be sent to an image inverter and digital magnifier 306. The image inverter and digital magnifier 306 may receive power from the power supply 340, either directly or through the power connection 336. The image inverter and digital magnifier 306 may invert the image from the black-and-white to color converter 304, so that it no longer appears upside down and backwards, as it did at the handheld magnifying lens 328.

The image inverter and digital magnifier 306 may also magnify the image from the black-and-white to color converter 304, so that particular areas of interest can be displayed in greater detail. The magnification of the image inverter and digital magnifier 306 may be controlled by a switch 310, which allows ophthalmologists to override the image inversion and keep the image as a virtual image. The image inverter and digital magnifier 306 may also include a zoom 308. The zoom 308 may control the degree of magnification of the image.

The image from the image inverter and digital magnifier 306 may be displayed on screens 318 and 324. In one embodiment, screens 318 and 324 are liquid crystal display (LCD) screens. In one embodiment, the screens 318 and 324 may slide medially and temporally in front of the ophthalmologist's eyes, to center the screens 318 and 324 on the virtual axes of the eyes. In one embodiment, the screens 318 and 324 may be mini high-resolution screens. The screens 318 and 324 may receive power from the power supply 340.

In front of the screens 318 and 324 may be placed diopter lenses 320 and 322, to refine and focus the image further. In one embodiment, the lenses 320 and 322 may limit the accommodation needed to focus on the screens 318 and 324.

An ophthalmologist wearing the wearable headset 338 may view the image of the eye to be examined 330 in a binocular fashion through the screens 318 and 324. This allows a stereo effect to be presented. If, for example, alternate images captured by the camera 312 and processed through the streaming video converter 302, the black-and-white to color converter 304, and the image inverter and digital magnifier 306 were presented to the screen 318 and the screen 324 in an alternating fashion, the eye may appear to have depth.

The depth effect would be produced by the movement of the ophthalmologist's head as it moves during the examination, allowing the camera 312 to see the eye from slightly different angles at different points in time. Taking an individual image captured by the camera 312 and presenting it to one of the eyes through the screen 318, and taking another individual image captured by the camera 312 and presenting it to the other eye through the screen 324 would have the effect of presenting images taken from slightly different angles to each other's eyes. Consequently, a stereoscopic effect would be produced by the separate images.

In one embodiment, the signal from the streaming video converter 302 may be sent to a computer 344, such as a laptop computer over a connection 342, for documentation and storage. In one embodiment, the lap top computer 344 may include a black-and-white to color converter 346 as well. The black-and-white to color converter 346 may convert the infrared image from the streaming video converter 302 to a visible image in the same manner as the black-and-white to color converter 304 described above.

Software 354 on the laptop computer 344 may be used to manipulate the image to adjust contrast, white balance, black balance, color saturation, and brightness, for example. Separate images from the camera 312 may be stitched together to form a montage. A three-dimensional image of an eye under examination may be developed, that can be rotated or translated in the x, y, or z axes.

Cross-sectional images can also be produced from the images on the laptop computer 344. The images can be digitally enhanced so that a particular tissue or vessel can be displayed more prominently, such as with an artificial color, to enhance visualization. Images of lesions or tumors can be measured and compared from sequential images taken during different patient visits. In one embodiment, the images may be stored as DICOM standard or MPEG-4 images.

In one embodiment, the images and files stored by the computer and transmitted by the video transmitter 360 will interface with hospital or medical record software systems, to allow downloads of the data to a medical record system. The laptop computer and/or the wearable headset 338 can be connected to a hospital or medical facility network over a wireless or a wired connection 356. Software on the lap top could allow remote access to the images.

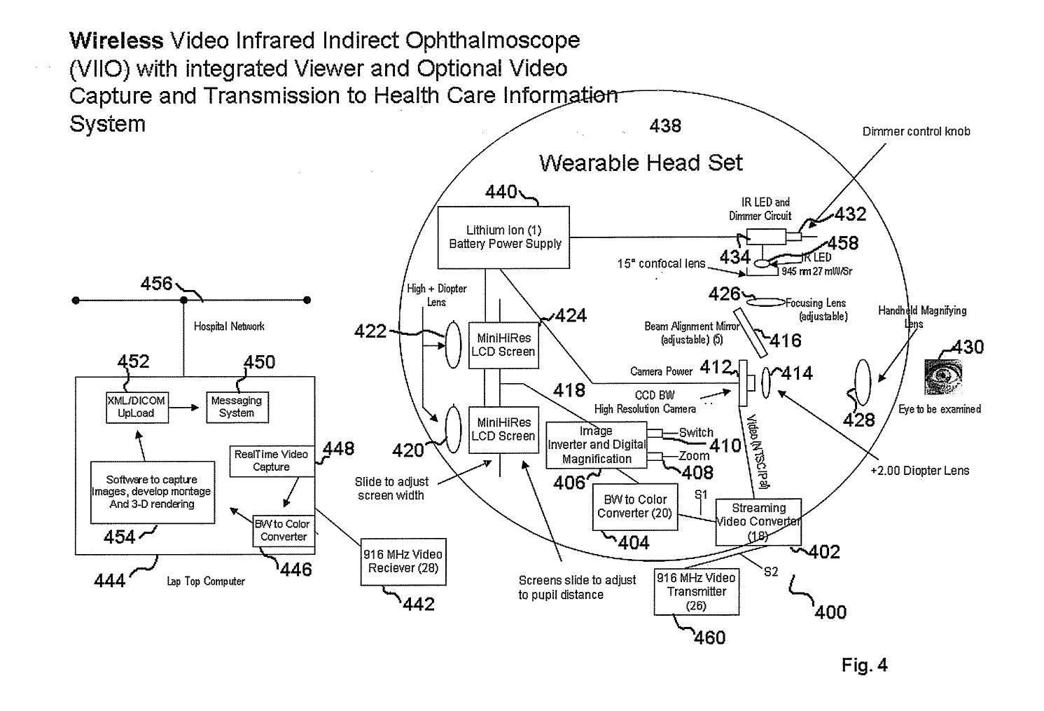

In FIG. 4 is shown a wireless indirect infrared opthalmoscope 400 according to an embodiment of the invention. In one embodiment, the indirect opthalmoscope 400 may be a binocular opthalmoscope. In this case, two viewing screens 418 and 424 could be used, one for each eye, as in a pair of binoculars. In the indirect opthalmoscope 400, light is not shined directly on the ocular system. Rather, the light is diverted by at least one reflector or beam splitter. The indirect opthalmoscope 400 may include a wearable headset 438. In one embodiment, an ophthalmologist places the wearable headset 438 on his head during the examination. Wearing the wearable headset 438 including the indirect opthalmoscope 400 on the head allows light to be directed on the point to be viewed, while leaving the hands free for other functions. A wearable headset 438, in particular, allows an ophthalmologist to direct the light by turning the head in the direction in which the ophthalmologist wants to look.

In another embodiment, the wearable headset 438 is a pair of glasses or goggles.

The ophthalmologist also holds a lens 428 to direct the light from the opthalmoscope onto a patient's eye to obtain a virtual image of the retina. The lens 428 focuses the light from the opthalmoscope. The light is directed into the patient's eye through the pupil, illuminating the retina inside the eye. The light reflected by the retina is reflected out of the patient's eye, and an indirect image is formed between the lens 428, which is held in front of the patient's eye, and the ophthalmologist's eye.

As shown in FIG. 4, an embodiment of a wearable headset 438 employs a light source 434 to illuminate the retina. The light source 434 may be an infrared light source, such as a light emitting diode (LED). In other embodiments, the light source 434 may be an electric lamp, a mercury vapor lamp, a halogen lamp, or a tungsten filament lamp. The light source 434 may be equipped with a filter to filter out visible wavelengths and pass infrared wavelengths of radiation. The light source 434 may be powered by a power supply 440. The power supply 440 may be a battery, such as a rechargeable lithium ion battery, a nickel cadmium battery, or an alkaline battery.

The light source 434 emits light of an invisible nature, i.e. light that is out of the visible range, and to which the eye is not generally sensitive. The infrared light may be of a wavelength in the range of 945 nm, for example. Since the light is invisible, the pupil does not constrict when it receives the infrared light, and the examination can be performed without artificial dilation. Thus, a patient who is allergic to eye drops does not need to have them put in their eyes. Also, since artificial dilation is not necessary to perform the examination with invisible light, an examination can be performed more quickly in, for example, an emergency situation.

The output of the light source 434 may be controlled by a dimmer circuit 432, such as a rheostat, or a solid-state device like a transistor. The ophthalmologist, for example, may be able to raise or lower the amount of infrared radiation emitted by the light source 434 during examination, as the need for infrared light changes.

In one embodiment, the light source 434 is a light emitting diode. Since light emitting diodes are current devices, the degree to which they illuminate is proportional to the amount of electric current flowing through the light emitting diode, rather than to the voltage drop across the light emitting diode. Consequently, a power supply that varies voltage across the light source 434 may not be efficient or linear when trying to control the intensity of radiation emitted from light source 434. In one embodiment, the intensity of radiation produced by the light source 434 is controlled by supplying a pulsed width square wave to turn the light source 434 on and off very rapidly. Since a light emitting diode has a very fast turn on time, typically measured in nano-seconds, the intensity of radiation emitted from the light source 434 can be varied by varying the width of the pulses supplied to the light source 434.

The light source 434 might also comprise a lamp, such as a halogen lamp that emits light in other wavelengths besides infrared. In this case, the light source 434 might include a filter that filters the wavelengths outside of the infrared band so that they do not reach the pupil and cause it to constrict. In one embodiment, the filter might take the form of a beam splitter that reflects visible radiation while allowing infrared radiation to pass.

The infrared radiation from the light source 434 may pass through a lens 458, which may be a confocal lens, and an adjustable diopter focusing lens 426 to obtain a uniform beam of light. In one embodiment, the lens 458 is a 10°-35° confocal lens. In one embodiment, the focusing lens 426 may be adjustable, for varying a focal length and focusing the infrared radiation on a beam splitter 416. The beam splitter 416 may be adjustable as well, reflecting the infrared radiation through the handheld magnifying lens 428 toward the eye to be examined 430. In one embodiment, the beam splitter 460 can be adjusted to move the light beam up and down, to focus the light on the eye to be examined 430.

In one embodiment, the beam splitter 416 may be a filter, passing visible radiation and reflecting infrared radiation through the handheld magnifying lens 428 and toward the eye to be examined 430. In another embodiment, the beam splitter 416 may be a 50/50 filter, passing half of the infrared radiation while reflecting the other half of the infrared radiation through the handheld magnifying lens 428 and toward the eye to be examined 430. This may be useful if, for example, the light source 434 emits more infrared radiation that is necessary for the examination. Other proportions than 50/50 may be used, the beam splitter could be a 60/40 filter, or a 70/30 filter, as well, and in the reverse proportions too.

A virtual image is produced by the handheld lens 428 between the handheld lens 428 and the wearable headset 438. The image is captured by the camera 412, after being focused on the camera 412 by a positive diopter lens 414, such as a positive 20 diopter lens.

The camera 412 may be powered by the power supply 440, either directly or through the power connection 436. The camera 412 may collect the infrared radiation reflected by the eye to be examined, and form an image, such as a black-and-white image. In one embodiment, the camera 412 could be a charge coupled device (CCD). In another embodiment the camera 412 is a complementary metal-oxide-semiconductor (CMOS) based device, or an array of LEDs working in reverse, i.e., collecting light and converting it into an electrical signal. In one embodiment, the camera 412 could be a high-resolution camera.

The images formed by the camera 412 may be sent to a streaming video converter 402. The streaming video converter 402 may receive power from the power supply 440, either directly or through the power connection 436. The streaming video converter may process the images from the camera 412, creating a streaming video from individual still images. In one embodiment, the images from the camera 412 are converted from an analog signal to a digital signal in the streaming video converter 402. In another embodiment, camera 412 is a digital CCD camera, and so no digital conversion is necessary. In one embodiment, the streaming video converter 402 may include a computer processor to perform the analog to digital conversion, and other processing as necessary. The analog to digital conversion may be performed in software.

From the streaming video converter 402 the images may be sent to a black-and-white to color converter 404. In an alternative embodiment, the images from the camera 412 may be sent to the black-and-white to color converter 404 directly. The black-and-white to color converter 404 may receive power from the power supply 440, either directly or through the power connection 436.

In one embodiment, the black-and-white to color converter 404 maps intensities of pixels of a charge coupled device to separate colors. Mapping the intensities of the pixels to colors may include interpolating pixel intensities between two (or more) pixels, or extrapolating pixel intensities around edges.

In one embodiment, the black-and-white to color converter 404 creates a map of grey scale to color that is appropriate for the various eye structures, like blood vessels, the optic nerve, and fovea. In this embodiment, the black-and-white to color converter 404 may normalize the black-and-white image of the eye. The image of the eye may be normalized with a histogram normalizer. Normalizing the image of the eye produces a uniform intensity profile of the image. The black-and-white to color converter 404 may also use edge detection image processing to identify the blood vessels and other structures of the eye. Finally, after the image of the eye has been mapped, direct spatial domain intensity transformations are applied to each structure of the eye, resulting in a colorized image of the eye.

The black-and-white to color converter 404 may, in another embodiment, scale the various wavelengths of infrared light represented by the image from the streaming video converter 402, so that wavelengths within the visible range are formed. Wavelengths in the range of about 480 to 750 nm, for example, may be visible to the human eye. From these scaled wavelengths a visible image could be formed, in which colors were assigned to various features of the eye under examination. In one embodiment, shades of gray are assigned to the scaled wavelengths, creating a grayscale image. In another embodiment, colors of the visible spectrum are assigned to the scaled wavelengths.

If the light source 434 produces light in the near infrared range, of slightly longer wavelengths than 750 nm, for example, the various wavelengths of light composing the infrared image could be scaled by a suitable scalar so they all entered the visible range in proportionally the same amount. Strictly by way of a non-limiting example, a wavelength of 945 nm, for example, in the infrared range could be scaled by 400 nm to 645 nm in the visible range. Consequently, a feature of the eye that reflects light at the wavelength of 945 nm would show up in the visible range as a particular “color.” Other wavelengths in the near infrared could be scaled in approximately the same amount. Light reflected by the feature in the range of about 950 nm, could be scaled in the same proportion as the 945 nm light, so that it assumes a wavelength of 650 nm in the visible range.

Images produced by the black-and-white to color converter 404 may be sent to an image inverter and digital magnifier 406. The image inverter and digital magnifier 406 may receive power from the power supply 440, either directly or through the power connection 436. The image inverter and digital magnifier 406 may invert the image from the black-and-white to color converter 404, so that it no longer appears upside down and backwards, as it did at the handheld magnifying lens 428.

The image inverter and digital magnifier 406 may also magnify the image from the black-and-white to color converter 404, so that particular areas of interest can be displayed in greater detail. The magnification of the image inverter and digital magnifier 406 may be controlled by a switch 410, which allows ophthalmologists to override the image inversion and keep the image as a virtual image. The image inverter and digital magnifier 406 may also include a zoom 408. The zoom 408 may control the degree of magnification of the image.

The image from the image inverter and digital magnifier 406 may be displayed on screens 418 and 424. In one embodiment, the screens 418 and 424 are liquid crystal display (LCD) screens. In one embodiment, the screens 418 and 424 may slide medially and temporally in front of the ophthalmologist's eyes, to center the LCD screen on the virtual axes of the eyes. In one embodiment, the screens 418 and 424 may be mini high-resolution screens. The screens 418 and 424 may receive power from the power supply 440.

In front of the screens 418 and 424 may be placed diopter lenses 420 and 422, to refine and focus the image further. In one embodiment, the lenses 420 and 422 may limit the accommodation needed to focus on the LCD screen.

An ophthalmologist wearing the wearable headset 438 may view the image of the eye to be examined 430 in a binocular fashion through the screens 418 and 424. This allows a stereo effect to be presented. If, for example, alternate images captured by the camera 412 and processed through the streaming video converter 402, the black-and-white to color converter 404, and the image inverter and digital magnifier 406 were presented to the screen 418 and the screen 424 in an alternating fashion, the eye may appear to have depth.

The depth effect would be produced by the movement of the ophthalmologist's head as it moves during the examination, allowing the camera 412 to see the eye from slightly different angles at different points in time. Taking an individual image captured by the camera 412 and presenting it to one of the eyes through the screen 418, and taking another individual image captured by the camera 412 and presenting it to the other eye through the screen 424 would have the effect of presenting images taken from slightly different angles to each other's eyes. Consequently, a stereoscopic effect would be produced by the separate images.

In one embodiment, the streaming video converter 402 may include a video transmitter 460. In one embodiment, the video transmitter 460 may transmit in the range of 800-1000 MHz, such as at 916 MHz. A signal from the streaming video converter 402 may be transmitted by the video transmitter 462 to a video receiver 442 coupled to a computer 444, such as a laptop computer, for documentation and storage. In one embodiment, the lap top computer 444 may include a black-and-white to color converter 446 as well. The black-and-white to color converter 446 may convert the infrared image from the video transmitter 462 a visible image in the same manner as the black-and-white to color converter 404 described above.

Software 454 on the laptop computer may manipulate the image to adjust contrast, white balance, black balance, color saturation, and brightness, for example. Separate images from the camera 412 may be stitched together to form a montage. A three-dimensional image of an eye under examination may be developed, that can be rotated or translated in the x, y, or z axes.

Cross-sectional images can also be produced from the images on the laptop computer 444. The images can be digitally enhanced so that a particular tissue or vessel can be displayed more prominently, such as with an artificial color, to enhance visualization. Images of lesions or tumors can be measured and compared from sequential images taken during different patient visits. In one embodiment, the images may be stored as DICOM standard or MPEG-4 images. In one embodiment, the images and files stored by the computer and transmitted by the video transmitter 460 will interface with hospital or medical record software systems, to allow downloads of the data to a medical record system. The laptop computer and/or the wearable headset 438 can be connected to a hospital or medical facility network over a wireless or a wired connection. Software on the lap top could allow remote access to the images.

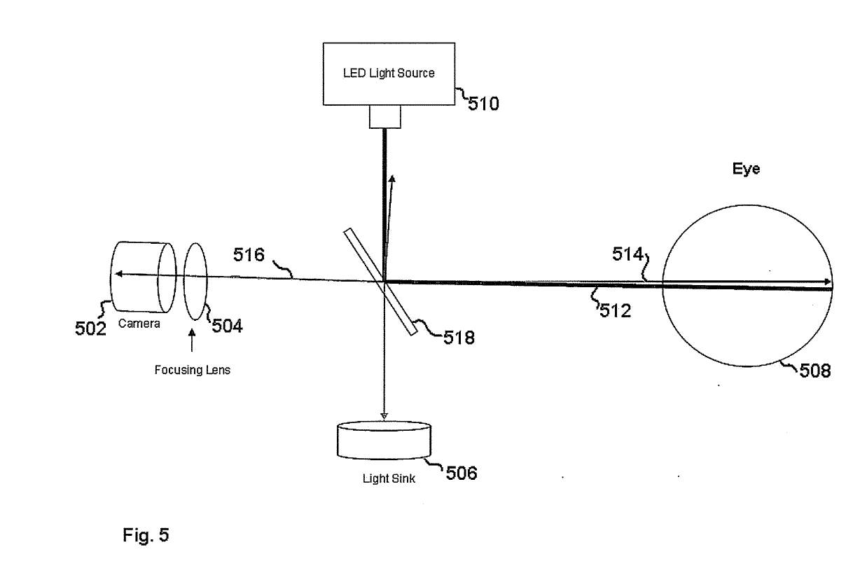

In FIG. 5 is shown a ray diagram for use with an infrared opthalmoscope. As may be seen in FIG. 5, infrared radiation emanating from an LED light source 510 is redirected by a beam splitter 518 toward an eye 508. The redirected beam is enumerated 514. The redirected beam of infrared radiation 514 reaches the eye 508 and is reflected off the eye 508 as beam 512. Beam 512 passes through the beam splitter 518 again as beam 516, and also passes through a focusing lens 504 before reaching a camera 502. Some of the infrared radiation from LED light source 510 passes through the beam splitter 518 and reaches light sink 506.

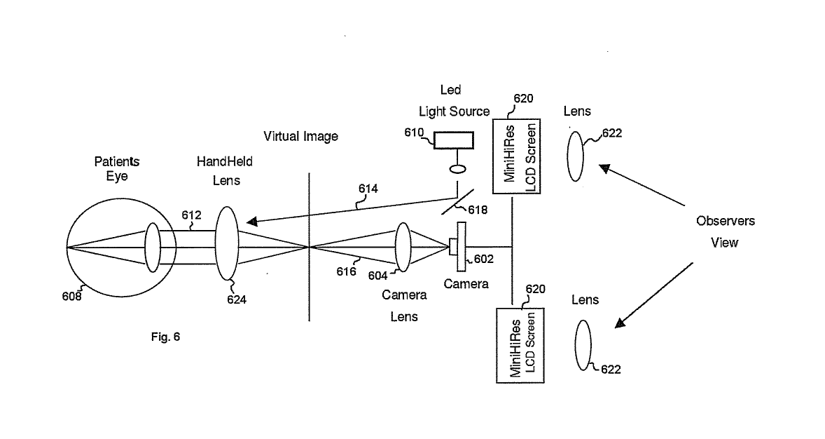

In FIG. 6 is shown a ray diagram for use with an infrared opthalmoscope. As may be seen in FIG. 6, infrared radiation emanating from of a light source 610 is redirected by a beam splitter 618 toward an eye 608. The redirected beam is enumerated 614. The redirected beam 614 may or may not pass through a handheld lens 624 held in front of the eyes 608 by the physician. The redirected beam 614 reaches the eye 608 and is reflected off of the eye 608 as beam 612, toward the handheld lens 624. The handheld lens 624 is used by the physician to focus the light on the eye 608 and eight in the examination. Beam 612 passes through the handheld lens 624 as beam 616 and is focused on a camera lens 604. A virtual image of the eye 608 is formed between the handheld lens 624 and the camera lens 604. The beam 616 passes through the camera lens 604 and is collected by a camera 602. A signal from the camera 602 is distributed to two display screen 620, in front of which may be placed lenses 622. Images of the eye 608 are displayed on the screens 622 and viewed by the physician.

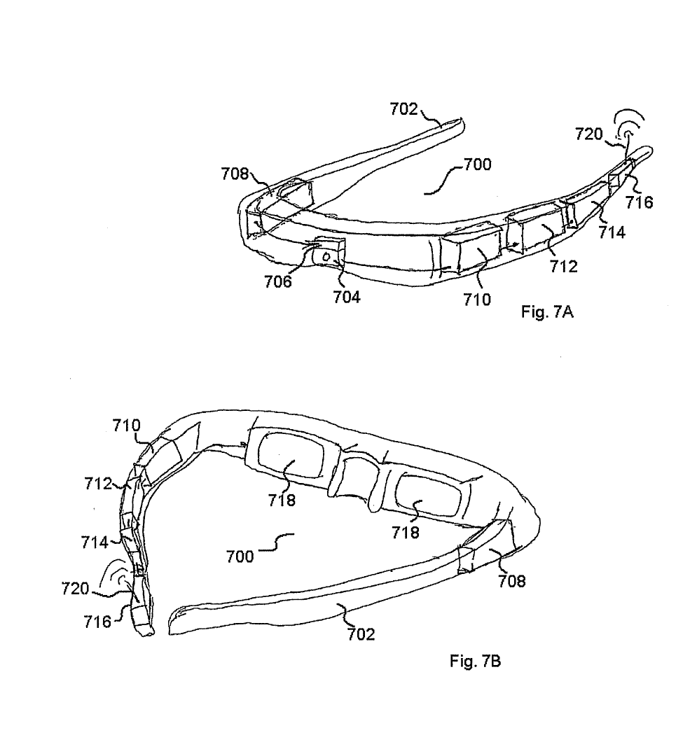

In FIGS. 7A and 7B is shown a wireless indirect infrared opthalmoscope 700 according to an embodiment of the invention. In this embodiment, the indirect infrared opthalmoscope is incorporated in a pair of glasses or goggles 702.

As may be seen in FIGS. 7A and 7B, the wireless indirect infrared opthalmoscope 700 includes an infrared light emitting diode 706. The infrared light emitting diode 706 may incorporate a confocal lens or a focusing lens. Infrared radiation from the infrared light emitting diode 706 is shed on an eye to be examined. The infrared radiation is reflected by the eye to be examined and is collected by a camera 704. The camera 704 and the infrared light emitting diode 706 may be powered by a power supply 708, such as a lithium ion battery power supply. A rheostat may be used to control the amount of power delivered to the infrared light emitting diode 706, and by implication, the light power emitted by the infrared light emitting diode 706.

The camera 704 may be, for example, a charge coupled device, or an array of light emitting diodes running in reverse, that is, collecting light, rather than emitting it. The camera 704 produces a signal representative of the infrared radiation it collects to a streaming video converter 710. The streaming video converter 710 may be powered by the power supply 708 as well. The streaming video converter 710 converts the signal from the camera 704 into video frames, and sends the video frames to a black-and-white to color converter 712. The black-and-white to color converter 712 may also be powered by the power supply 708.

The black-and-white to color converter 712 assigns colors to the various wavelengths of the infrared radiation collected by the camera 704 and incorporated in the video frames produced by the streaming video converter 710. The colors may be assigned, for example, by scaling various wavelengths of the infrared radiation by a predetermined amount, so that wavelengths in the visible range are created. Consequently, a feature of the eye that reflects one wavelength of infrared radiation may be seen to be distinct from another feature of the eye that reflects another wavelength of infrared radiation.

The color-converted video frames are sent from the black-and-white to color converter 712 to an image inverter and digital magnifier 714. The image inverter and digital magnifier 714 may be powered by the power supply 708. The image inverter and digital magnifier 714 inverts the image so it is right side up when viewed by an ophthalmologist or optometrist who is wearing the glasses 702. The image inverter and digital magnifier 714 also magnifies the view of the eye, allowing small details of the eye to be examined. The image inverter and digital magnifier 714 sends the converted and magnified image to a pair of display screens 718. The display screens 718 may be, for example, mini high-resolution liquid crystal displays. The display screens 718 may receive their power from the power supply 708 as well.

The image signal sent from the image inverter and digital magnifier 714 is displayed on the display screens 718, so that the image can be viewed by an ophthalmologist or optometrist wearing glasses 702.

The image signal from the image inverter into magnifier 714 may also be sent to a transmitter 716. The transmitter 716 may receive power from the power supply 708. The transmitter 716, which may include an aerial 720, may transmit the signal to a laptop computer or other network device, for review or storage of the images.

Transmitter 716 could, in the alternative, transmit the signal at an intermediate point, such as from the streaming video converter 710. In that case, the further processing of the signal, such as black-and-white to color conversion, image and version, and digital magnification, could be performed on a laptop accessible over a network by the transmitter 716.

Although a few preferred embodiments of the present invention have been shown and described, it would be appreciated by those skilled in the art that changes may be made in these embodiments without departing from the principles and spirit of the invention, the scope of which is defined in the claims and their equivalents.

What is claimed is:

1. An indirect opthalmoscope, comprising:
a wearable headset;
the wearable headset comprising a light source, a beam splitter reflecting infrared radiation from the light source to an eye, a camera collecting radiation reflected by the eye through the beam splitter, an analog to digital convertor receiving a raw signal from the camera based on the collected radiation, the analog to digital convertor converting the raw signal to a digital signal; a black and white to color converter converting the digital signal into a color signal, a streaming video converter processing the color signal into a video signal, and a pair of video monitors displaying an image of the eye based on the video signal;
the wearable headset further comprising a video transmitter, the video transmitter transmitting the video signal to a computer over a network, the computer extracting a plurality of images from the video signal.

2. The indirect opthalmoscope of claim 1, wherein the video monitors comprise high-resolution liquid crystal display screens.

3. The indirect opthalmoscope of claim 1, wherein the light source further comprises a rheostat dimmer circuit.

4. The indirect opthalmoscope of claim 1, wherein the light source further comprises an infrared filter and a focusing lens, the infrared filter substantially blocking visible and ultraviolet radiation, and the focusing lens focusing the infrared radiation from the light source on the beam splitter.

5. The indirect opthalmoscope of claim 1, further comprising a power supply to supply power to the light source, the power supply selected from the group consisting of a rechargeable lithium ion battery, a nickel cadmium battery, or an alkaline battery.

6. The indirect opthalmoscope of claim 1, wherein the network is selected from the group consisting of a wired network, and a wireless network.

7. The indirect opthalmoscope of claim 1, wherein the computer comprises:
a real-time video capture capturing images from the video signal, a black-and-white to color converter converting the images to color, 3-D rendering software, and a messaging system.

8. The indirect opthalmoscope of claim 1, wherein the wearable headset is selected from the group consisting of a pair of glasses and a pair of goggles.

9. The indirect opthalmoscope of claim 1, wherein the light source is selected from the group consisting of a light emitting diode, an electric lamp, a mercury vapor lamp, a halogen lamp, and a tungsten filament lamp.

10. The indirect opthalmoscope of claim 1, wherein the light source comprises a halogen lamp with an infrared pass filter, the infrared path filter substantially blocking visible and ultraviolet radiation.

11. The indirect opthalmoscope of claim 1, wherein the black and white to color converter maps intensities of grayscale pixels to colors.

12. A direct opthalmoscope, comprising:
a light source, a beam splitter reflecting infrared radiation from the light source through one of a plurality of focusing lenses to an eye, a camera collecting radiation reflected by the eye through the beam splitter, an analog to digital convertor receiving a raw signal from the camera based on the collected radiation, the analog to digital convertor converting the raw signal to a digital signal; a black and white to color converter converting the digital signal into a color signal, a streaming video converter processing the color signal into a video signal, and a video monitor displaying an image of the eye based on the video signal;
the direct opthalmoscope further comprising a video transmitter, the video transmitter transmitting the video signal to a computer over a network, the computer extracting a plurality of images from the video signal.

13. The direct opthalmoscope of claim 12, wherein the video monitor comprises high-resolution liquid crystal display screens.

14. The direct opthalmoscope of claim 12, wherein the light source further comprises a rheostat dimmer circuit, an infrared filter, and a focusing lens, the infrared filter substantially blocking visible and ultraviolet radiation, and the focusing lens focusing the infrared radiation from the light source on the beam splitter.

15. The direct opthalmoscope of claim 12, further comprising a power supply to supply power to the light source, the power supply selected from the group consisting of a rechargeable lithium ion battery, a nickel cadmium battery, or an alkaline battery.

16. The direct opthalmoscope of claim 12, wherein the network is selected from the group consisting of a wired network, and a wireless network.

17. The direct opthalmoscope of claim 12, wherein the computer comprises:
a real-time video capture capturing images from the video signal, a black-and-white to color converter converting the images to color, 3-D rendering software, and a messaging system.

18. The direct opthalmoscope of claim 12, wherein the light source is selected from the group consisting of a light emitting diode, an electric lamp, a mercury vapor lamp, a halogen lamp, and a tungsten filament lamp.

19. The direct opthalmoscope of claim 12, wherein the light source comprises a halogen lamp with an infrared pass filter, the infrared path filter substantially blocking visible and ultraviolet radiation.

20. The direct opthalmoscope of claim 12, wherein the black and white to color converter maps intensities of grayscale pixels to colors.

21. A method of scanning an eye, comprising:
providing a light source;
emitting infrared radiation from the light source toward a beam splitter;
reflecting the infrared radiation with the beam splitter through a focusing lens;
focusing the infrared radiation with the focusing lens on the eye;
collecting radiation reflected by the eye through the beam splitter at a camera;
producing an image signal representative of an image of the eye with the camera based on the collected radiation; and
displaying the image of the eye produced by the image signal on a display.

22. The method of scanning an eye of claim 21, further comprising transmitting the image of the eye over a network to another computer.
